(19) United States
(12) Patent Application Publication
(10) Pub. No.: US 2012/0203199 A1
(43) Pub. Date: Aug. 9, 2012

(54) Method and apparatus for providing access to an internal body organ

(75) Inventors: Alfred E. Mann, Las Vegas, NV (US); Gregory M. Harbers, Valencia, CA (US); Kelly A. Chapman, Tuscon, AZ (US); Abram D. Janis, Elkridge, MD (US)

(73) Assignee: INCUMED, LLC, VALENCIA, CA (US)

(21) Appl. No.: 13/373,787

(22) Filed: Nov. 30, 2011

Related U.S. Application Data
(60) Provisional application No. 61/458,784, filed on Nov. 30, 2010.

(51) Int. Cl. A61M 5/00; A61M 29/00; A61B 17/34; A61M 25/01

(52) U.S. Cl. 604/506; 604/171; 604/96.01; 604/272; 604/164.01; 604/170.01; 604/164.1

(57) ABSTRACT

Catheter systems and methods for facilitating the delivery of a therapeutic agent directly to the interior of a body organ, and/or the extraction of material from said organ. The catheter distal end is placed within the body organ to be treated. The catheter may carry a fixation device near the catheter distal end. When implanted, the fixation device retains the catheter distal end in the organ despite normal patient activity. The fixation device may include porous material to promote soft tissue ingrowth for anchoring the catheter distal end within the organ.

### CROSS-REFERENCE TO RELATED APPLICATION

This application claims the benefit of U.S. Provisional Application Ser. No. 61/458,784, filed Nov. 30, 2010 and entitled “Method and Apparatus for Providing Long-Term Access to an Internal Body Organ,” which is incorporated herein by reference in its entirety.

### FIELD OF THE INVENTION

This application relates generally to medical technology and more particularly to methods and apparatus for providing long term repetitive or continuous access to an internal body organ, e.g., a solid body organ. Embodiments of the invention are useful in a variety of medical procedures, e.g., for delivering a therapeutic agent to the interior of a lymph node.

### BACKGROUND OF THE INVENTION

Efforts to treat cancer with vaccines date back to the origins of immunology. Numerous approaches to stimulate the immune system to recognize tumors have been tried over the years. Vaccines consisting of peptide or protein administered with an adjuvant have been the most frequently used. These adjuvants include compounds such as bacterial cell wall components that incite an inflammatory response, or cytokines such as interleukin-12 (IL-12), or granulocyte-macrophage colony stimulating factor (GM-CSF). Monocytes, neutrophils, eosinophils, and T-cells are all recruited to the site of the inflammation where the adjuvant is used. However, only a transient cytolytic T-cell response is generated to respond to the antigens presented by the local professional antigen-presenting cells (APCs), during administration of a vaccine via subcutaneous or intradermal route.

A number of studies have focused on the option of introducing vaccines directly into the lymph nodes of patients. Preclinical experiments in mice have shown that an intranodal injection of a dendritic cell vaccine was superior to intravenous, subcutaneous or intramuscular routes for the generation of a potent immune response. In order to counteract the generation of only a transient T-cell response, repeated intranodal infusions of the vaccines over a short period of time have been tried. The goal is to create an immunogenic scenario similar to that seen during a bacterial or viral infection, whereby a high antigen uptake by APCs leads to high levels of cytolytic T-cells, leading to a longer and more effective response against the antigens.

Multiple patent and scientific publications discuss various aspects of directly introducing a therapeutic agent into a solid body organ, e.g., a lymph node. For example only, note Patent Publication US 2008/0014211 which states:
“While the poor pharmacokinetics of free peptides has prevented their use in most routes of administration, direct administration into secondary lymphoid organs, particularly lymph nodes, has proven effective when the level of antigen is maintained more or less continuously by continuous infusion or frequent (for example, daily) injections.”

Such procedures requiring repetitive percutaneous injections of vaccine, peptides and other therapeutic agents into organs for extended periods can, as a practical matter, prove to be quite cumbersome and inconvenient, thus posing a significant challenge to both the operating physician and the patient.

### SUMMARY OF THE INVENTION

The present inventions relate to methods and apparatus for providing access, such as long term repetitive or continuous access, to an internal body organ, e.g., a solid body organ. More particularly, the invention relates to catheter systems and methods for facilitating the delivery of one or more therapeutic agents directly to the interior of a body organ, and/or the extraction of material from said organ.

Embodiments of the invention are useful in a variety of procedures for delivering or extracting material to and from a body organ, e.g., a solid body organ. Such organs typically shift in position relative to an exterior anchor point as a patient moves his body, thus making it difficult to reliably retain a catheter distal end in an organ for an extended period, e.g., one year. Such organs can include, but are not limited to, the following: lymph nodes, glands, liver parenchyma, kidney parenchyma, spleen, pancreas, testicle, ovary, brain, skin (full thickness), lung stroma, muscle (skeletal, smooth, and cardiac), bone, nerves, and the walls of organs with lumen (gall bladder, arteries, veins, large lymph vessels, intestines, stomach, trachea, bronchioles).

An apparatus in accordance with some embodiments of the invention includes a flexible elongate catheter adapted to be percutaneously implanted in a patient in a manner to place the catheter distal end within the periphery of a body organ to be treated. The catheter carries a fixation device near the catheter distal end. When implanted, the fixation device retains the catheter distal end in place in the organ despite normal patient activity that may produce relative movement between the organ and the catheter proximal end anchor point. The catheter can extend percutaneously allowing for the catheter proximal end to be anchored to the external surface of the patient's skin. In other embodiments, the catheter is fully subcutaneous and the proximal end can be coupled to a subcutaneously implanted device having a closed chamber. The chamber may include a penetrable self sealing septum to allow needle access to the chamber.

A fixation device in accordance with some embodiments of the invention includes porous material adapted for subcutaneous placement just outside the internal organ to promote soft tissue ingrowth for anchoring the catheter distal end within the organ and for maintaining it stable over an extended period, e.g., one year or more. The fixation device may comprise, e.g., a tubular member, or sleeve, formed of porous material and mounted on and adhered to the outer surface of the catheter. The fixation device distal boundary may be positioned close to the catheter distal end, e.g., within 0.5 inch of the catheter distal end. The fixation device may be formed of porous material, e.g., titanium mesh, and configured to promote soft tissue ingrowth or integration and to and prevent tissue injury or infection. For example, the porous material may be characterized by a pore size between 50 and 200 microns with a porosity of 60% to 90%.

In some embodiments of the invention, a dilation member is mounted on the catheter distally of the fixation device. The dilation member may comprise a nose cone, or frustum of a cone, having an outer peripheral surface that increases from a diameter D1 near the catheter distal end to a diameter D2 near the sleeve distal end where D2 is equal to or greater than the diameter D3 of the porous material.

Some apparatus in accordance with the invention further include a first, or insertion, needle comprising a shaft having a sharp tip distal end for penetrating body tissue. The shaft distal end may be formed of echogenic material which can be readily imaged by ultrasonic, fluoroscopic, or other appropriate techniques, to facilitate precise placement of the shaft distal end in the organ of interest. The insertion needle is dimensioned for linear movement through an elongate lumen extending axially through the catheter with the needle sharp tip distal end extending slightly beyond the catheter distal end, for penetrating the organ peripheral wall. The insertion needle may be placed in the catheter prior to implanting to minimize the possibility of the sharp tip damaging the catheter. In the course of implanting, the operating physician will use the sharp tip to pierce the organ periphery to allow the catheter distal end to enter and be positioned within the organ. The insertion needle can then be withdrawn from the catheter, leaving the catheter distal end in place.

Although the insertion needle could be used for delivering a therapeutic agent to the organ, in other embodiments, the insertion needle is withdrawn once the catheter distal end has been placed. This helps avoid unintended injury to the patient or catheter. Further, although the catheter lumen could be used directly as the delivery path, in yet other embodiments, a second, or delivery, needle is used. This helps to assure penetration of any blockage or fouling formed at the catheter distal end. The delivery needle may comprise a shaft having an axial lumen therein extending from a proximal end to an open blunt tip distal end. In use, the blunt tip distal end is passed through the catheter lumen to place the blunt tip beyond the catheter distal end within the body organ. A therapeutic agent can then be delivered into the delivery needle proximal end, and from there, via the lumen shaft into the interior of the body organ. Additionally or alternatively, material can be extracted from the organ for removal or examination at the delivery needle shaft proximal end.

The catheter proximal end may be provided with a connector such as a female-luer connector, adapted to mate with a cooperating connector, e.g., a male luer connector, mounted on the proximal ends of the insertion and delivery needles.

### BRIEF DESCRIPTION OF THE FIGURES

FIG. 1 is a schematic representation of a percutaneous catheter, in accordance with some embodiments of the invention, for providing access to an internal body organ;

FIG. 2A is an isometric view of a catheter and insertion needle assembly in accordance with some embodiments of the invention, FIG. 2B is a side view of the assembly of FIG. 2A, and FIG. 2C is an enlarged view depicting the distal end portion of the assembly;

FIG. 3A is an isometric view of the catheter of FIG. 2A, and FIGS. 3B, 3C, and 3D, respectively, show side, rear and front views of the catheter;

FIG. 4A is a side view of the fixation device of FIG. 2A, while FIGS. 4B and 4C are respective isometric views of the fixation device, FIGS. 4D and 4E are, respectively, rear and front views of the fixation device, and FIG. 4F is a longitudinal sectional view through the fixation device of FIG. 4A;

FIG. 5A is an isometric view of the insertion needle of FIG. 2A, and FIGS. 5B, 5C, and 5D, respectively, show side, rear and front views of the insertion needle;

FIG. 6A is an isometric view of a delivery needle in accordance with some embodiments of the invention, while FIGS. 6B and 6C are side views of the delivery needle, FIGS. 6D and 6E, respectively, show rear and front views of the delivery needle, and FIG. 6F is an enlarged view of the distal tip of the delivery needle;

FIG. 7A is an isometric view showing the delivery needle of FIG. 6A mounted within the catheter of FIG. 3A, while FIGS. 7B and 7D are respective side views of this catheter/delivery needle combination, FIG. 7C is an enlarged view of the distal end portions of the catheter and delivery needle, and FIGS. 7E and 7F, respectively, show rear and front views of the catheter/delivery needle combination;

FIG. 8A is an isometric view of an alternative catheter construction employing an alternative fixation device, and FIGS. 8B, 8C, and 8D, respectively, show side, rear, and front views of the catheter of FIG. 8A;

FIG. 9A is a side view of the fixation device of FIG. 8A, while FIGS. 9B and 9C are respective isometric views of this fixation device, FIGS. 9D and 9E are respective rear and front views of the fixation device, and FIG. 9F is a longitudinal sectional view through the fixation device of FIG. 9A;

FIGS. 10A-10I schematically depict an exemplary sequence of steps involved in using an apparatus in accordance with some embodiments of the invention for introducing a therapeutic agent directing into an internal body organ;

FIG. 11 is a schematic illustration of an alternative embodiment of the invention employing a subcutaneously implanted device having a penetrable self sealing septum; and

FIG. 12 is a schematic illustration of a further alternative employing an external device coupled to the catheter proximal end.

### DETAILED DESCRIPTION

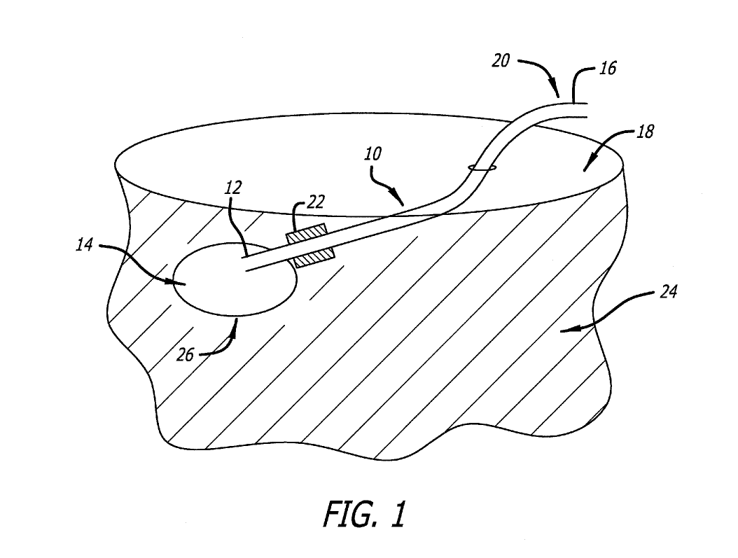

Attention is initially directed to FIG. 1 which schematically illustrates an indwelling catheter 10 in accordance with some embodiments of the invention, implanted so as to locate its distal end 12 within a body organ 14, e.g., a lymph node solid body organ. As previously mentioned, the medical literature discusses the benefits of providing direct access to the interior of a body organ over an extended period, e.g., one year or more, for performing a continuous or repetitive regimen, e.g., delivering a therapeutic agent or draining a body fluid. Because an organ 14 can move in the course of normal patient activity relative to the catheter proximal end portion 16, which is typically anchored to the patient's skin 18 at 20, a catheter in accordance with the present invention includes a fixation device 22 mounted on the catheter outer surface for retaining the catheter distal end 12 in the organ 14. The fixation device 22 may be mounted, e.g., within 0.5 inch of the catheter distal end 12, so that when the catheter is implanted, the fixation device 22 is located in the patients subcutaneous tissue 24 just outside the peripheral wall 26 of organ 14. As will be discussed further hereinafter, a fixation device 22 may include porous material configured to promote tissue ingrowth for providing a good subcutaneous anchor point and for minimizing the likelihood of tissue injury or infection.

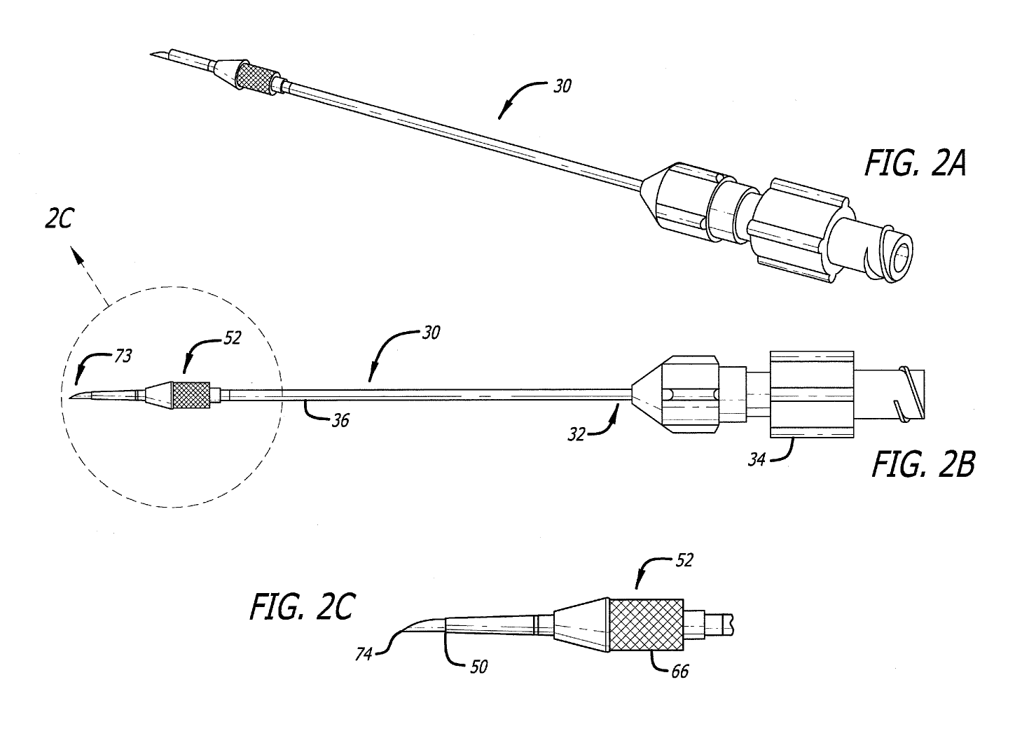

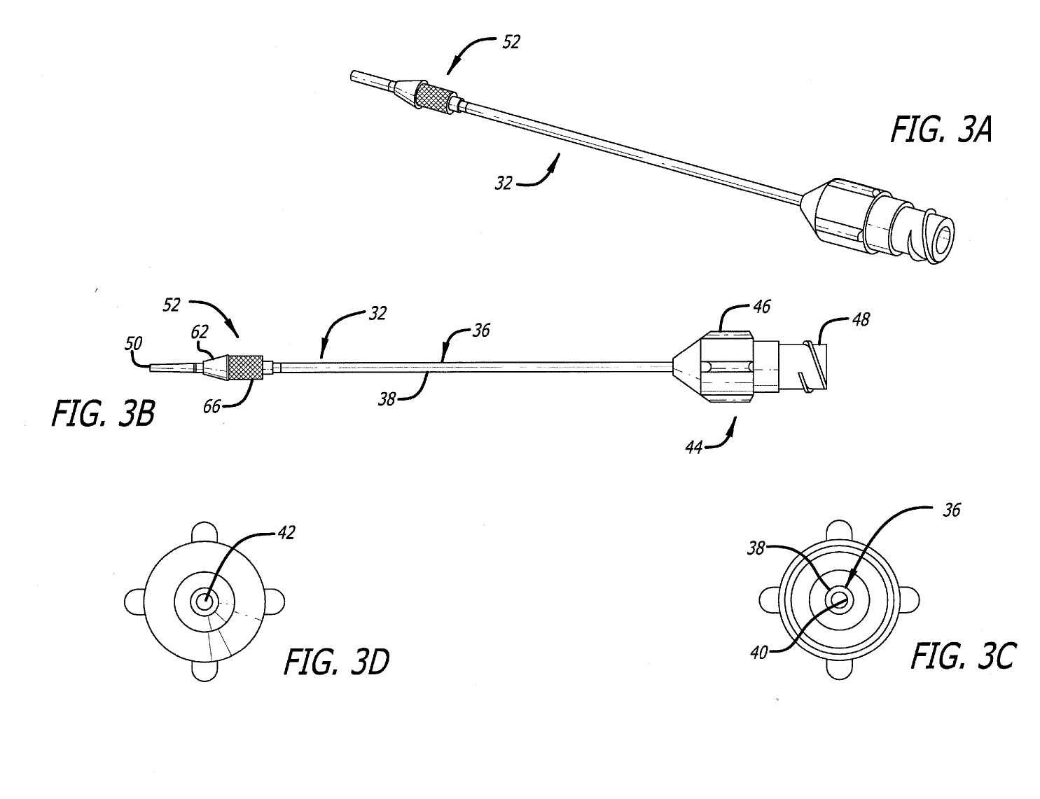

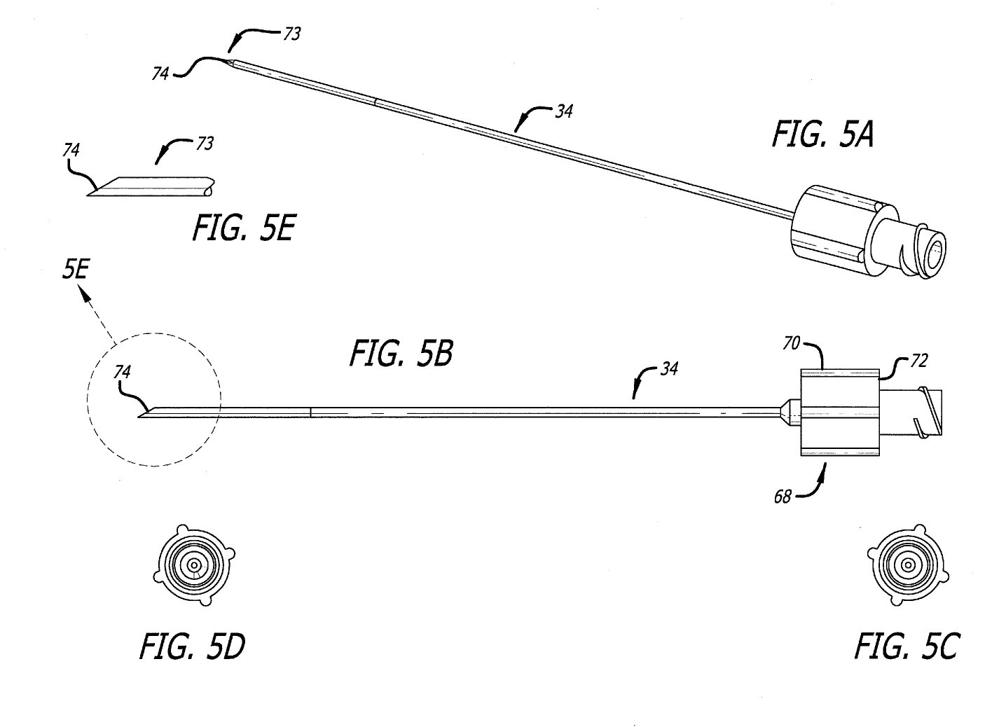

Attention is now directed to FIGS. 2A-2C which depict a catheter assembly 30, in accordance with some embodiments of the invention, for providing access to an internal body organ. The assembly 30 is comprised of a delivery catheter 32 (shown in detail in FIGS. 3A-3D) configured to accommodate an insertion needle 34 (shown in detail in FIGS. 5A-5E). Several of the Figures (e.g., 3B, 5B) show dimensions for embodiments intended for long term access to lymph nodes. It should be understood that these dimensions are only exemplary and can vary considerably, particularly dependent on the intended application, e.g., for accessing different body organs.

The delivery catheter 32 (FIGS. 3A-3D) comprises an elongate flexible tube 36 defining an outer peripheral surface 38 and an inner peripheral surface 40 surrounding a central lumen 42. The catheter 32 has a proximal end portion 44 that may carry a connector 46, e.g., incorporating a female luer connection 48. The lumen 42 extends from the proximal end portion 44 to a distal end 50 which is intended for implantation within the organ 14 as represented in FIG. 1.

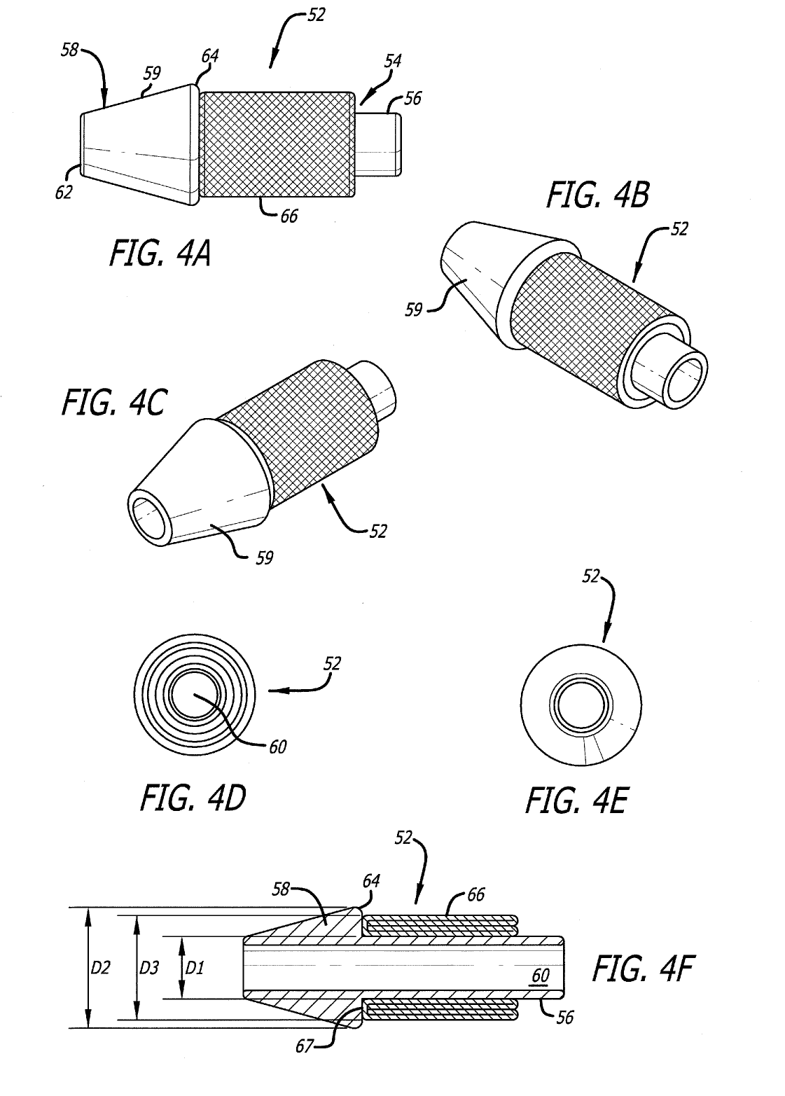

The catheter outer surface 38 has a fixation device 52 fixedly mounted thereon near the catheter distal end 50. A fixation device 52 (FIGS. 4A-4F) according to some embodiments includes a base member 54 defining a proximal sleeve portion 56 and a distal dilator portion 58. The catheter 32 extends through a bore 60 in the base member 54 enabling the base member to be fixedly adhered to the catheter outer surface 38, e.g., via appropriate medical adhesive or molding techniques. The dilator portion 58 may define a cone, or a frustum of a cone, having a smooth outer surface 59 whose diameter increases from D1 at its distal end 62 to D2 at a shoulder 64 adjacent the distal boundary of the sleeve portion 56. The base member 54 is mounted on the catheter, as described earlier, with the dilator distal end 62 located, e.g., within about 0.4 inch of the catheter distal end 50 (FIG. 3B).

As mentioned earlier, in some embodiments, a sleeve 66 formed of porous material, e.g. titanium mesh, is mounted around and adhered to at least a section of the outer surface of the base member sleeve portion 56. The sleeve porous material is selected to promote soft tissue in-growth and may be characterized by a pore size and density, e.g., a pore size between 50 and 200 microns and a pore density of between 60% and 95%. The sleeve 66 defines a diameter D3 where D3 is equal to or less then D2. The sleeve 66 is mounted on at least a section of the sleeve portion 56 so that its distal boundary 67 is located, e.g., within 0.5 inch of the catheter distal end 50.

FIGS. 5A-5E illustrate in detail the insertion needle 34 of FIGS. 2A-2C. The insertion needle has an outer diameter dimensioned to be accommodated within central lumen 42 of catheter 32. The proximal end 68 of the insertion needle 34 carries a connector 70, e.g., a male luer connector 72, configured to mate with catheter connector 48. The distal end 73 of needle 34 is configured with a tip 74 sharp enough to facilitate cutting through subcutaneous tissue and the peripheral wall of organ 14. The insertion needle 34 shaft length is dimensioned to be longer than the delivery catheter 32 so that when the needle is accommodated in the catheter with the insertion needle connector 72 and catheter connector 48 mated, the needle tip 74 will extend beyond the catheter distal end 50 (FIG. 2C).

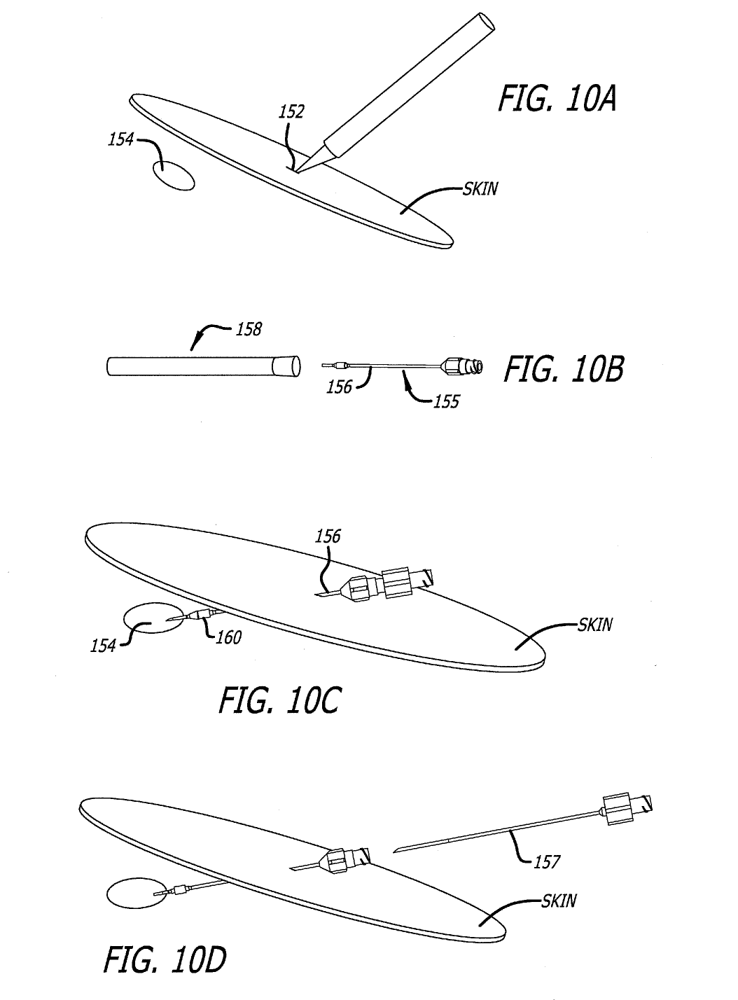

Some or all of insertion needle distal end 73, including some or all of tip 74, may include echogenic material for enabling an operating physician utilizing known imaging techniques, such as ultrasound or fluoroscopic x-ray-guidance or the like, to accurately place the tip 74 and catheter distal end 50 within the organ 14. Once placed, the insertion needle can be withdrawn from the catheter, leaving the catheter distal end 50 within the organ 14. The distal end 50 is placed in the organ 14 so as to locate the fixation device 52 in the patient's subcutaneous tissue just outside of the organ peripheral wall 26 (FIG. 1). As will be discussed further hereinafter, a temporary end cap 150 (FIG. 10E) may be placed over the catheter proximal end (e.g., secured to connector 44), then the catheter proximal end and connector 44 can be temporarily anchored to the patient's skin by appropriate dressing and tape. Over time, the patient's subcutaneous tissue will grow into and integrate with the fixation device porous material to assure retention of the catheter distal end 50 within the organ while permitting the patient to resume relatively normal activity which may involve relative movement between the organ and catheter external anchor point.

With the catheter distal end 50 retained within the organ 14 and the connector 44 readily accessible, a conventional syringe can be coupled to the connector to directly introduce a therapeutic agent into the organ 14 or extract/drain material from the organ via catheter lumen 42.

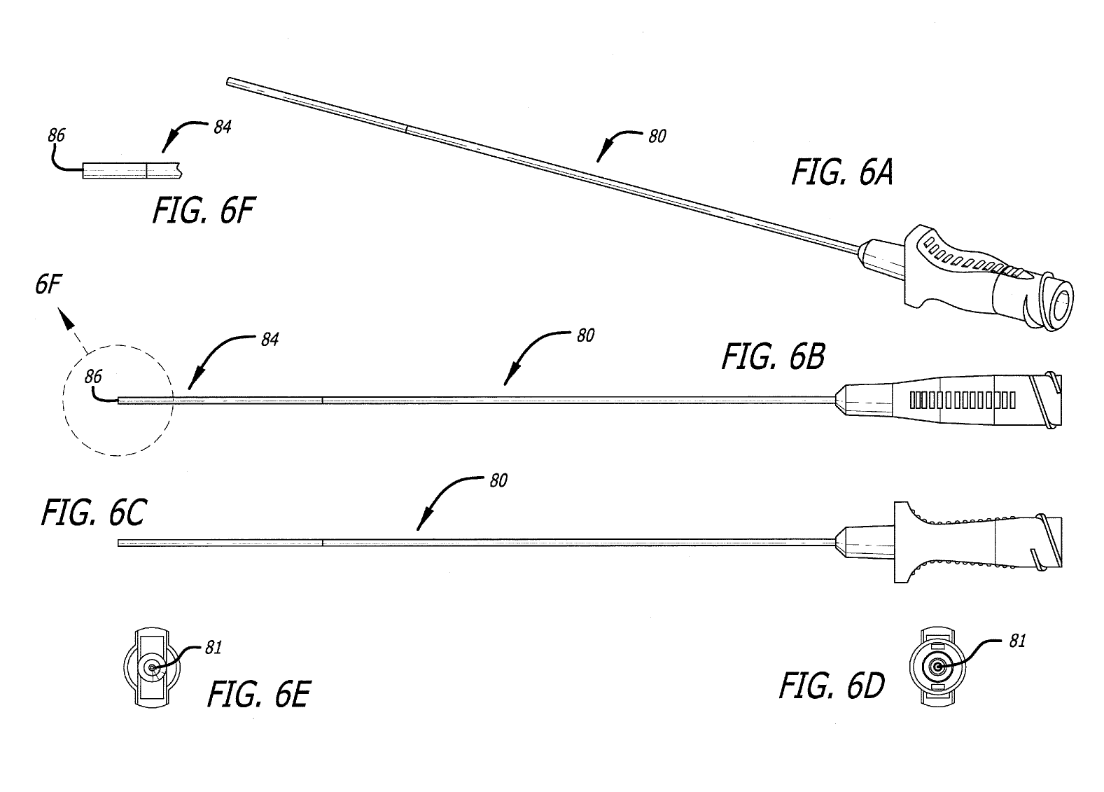

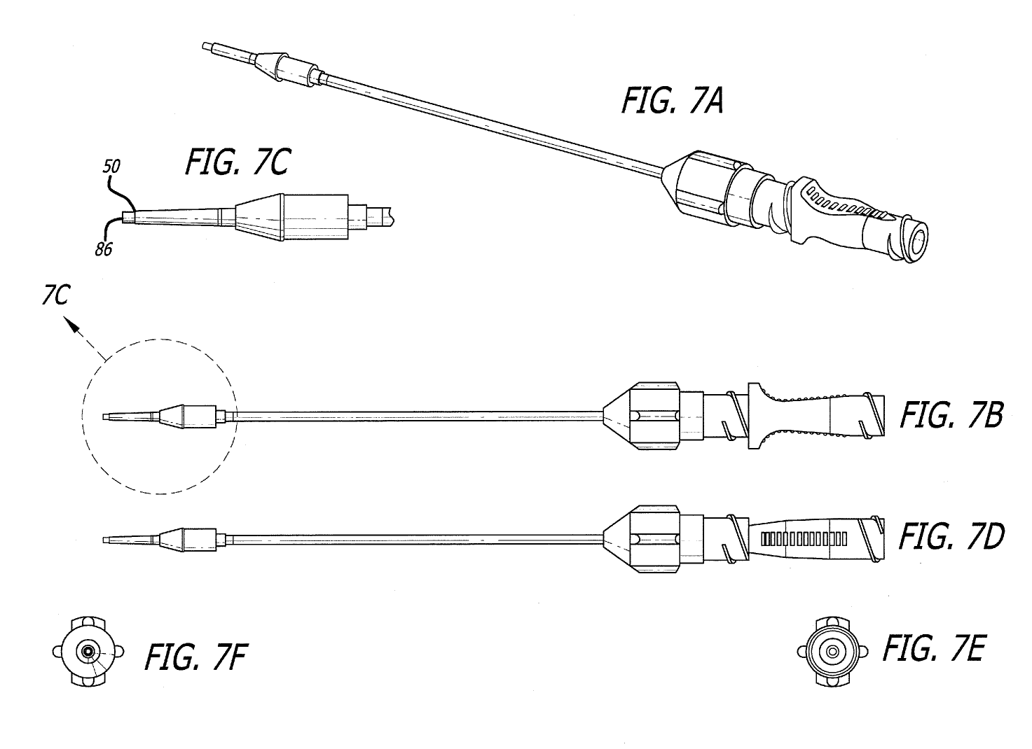

However, in other embodiments of the invention, in lieu of using the catheter lumen 42 for directly transporting material, a delivery needle 80 (FIGS. 6A-6F) having a lumen 81 is provided for accommodation within the catheter lumen 42 (FIGS. 7A-7F), similar to the aforediscussed insertion needle 34. In contrast to the insertion needle, the delivery needle distal end 84 includes a blunt tip 86 which facilitates penetration of any blockage or fouling which can form at the catheter open distal end 50 and which reduces the risk of injury to the patient or catheter as the delivery needle 80 is passed through catheter connector 44 and lumen 42. The delivery needle 80 is dimensioned so that when properly installed in the catheter 32 (FIGS. 7A-7F), the needle blunt tip 86 will extend past the catheter distal end 50 (FIG. 7C).

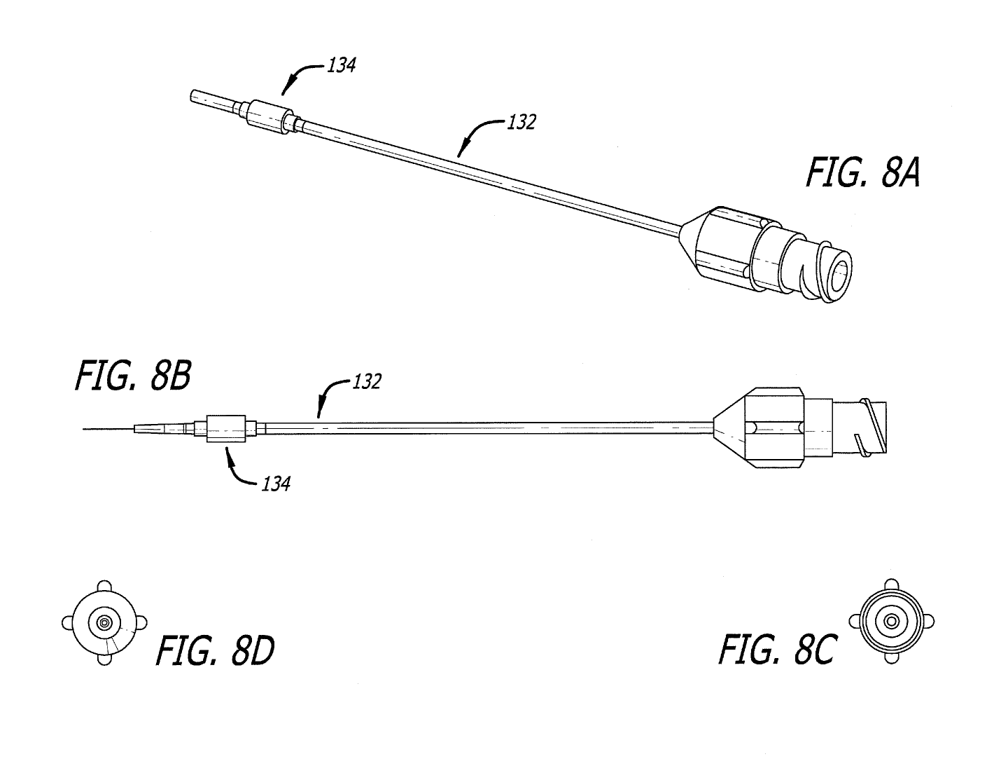

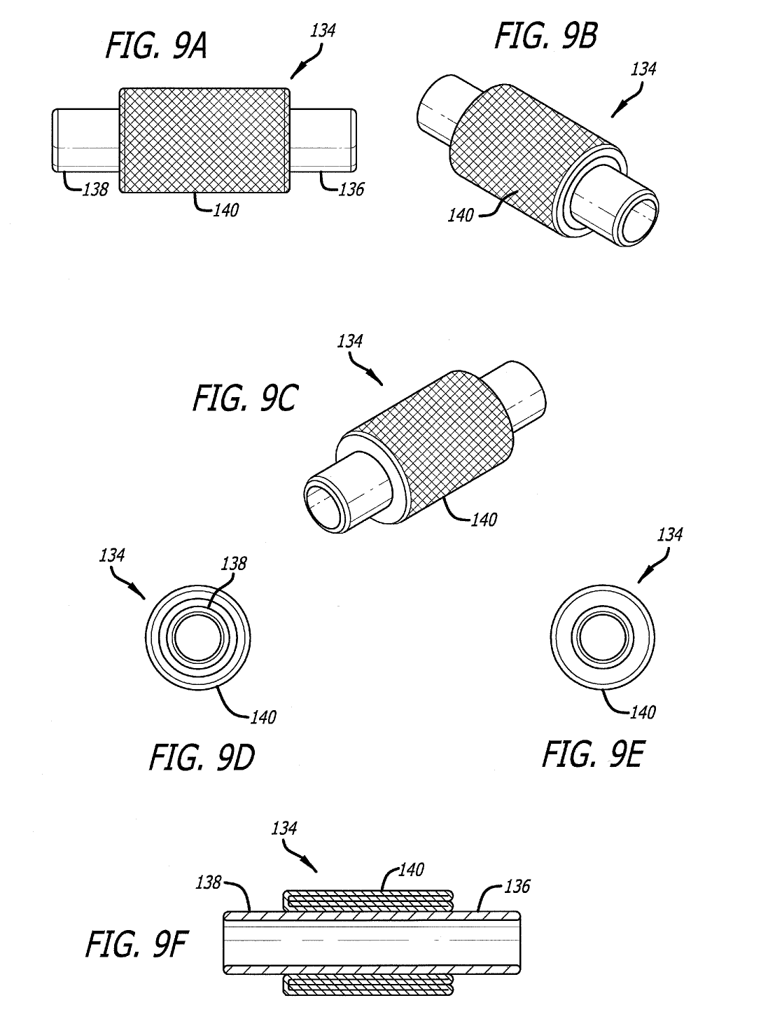

FIGS. 8A-8D depict an alternative delivery catheter 132 which is similar to the aforediscussed catheter 32 (FIGS. 3A-3D) except that it carries a modified fixation device 134 which differs from the fixation device 52 shown in FIGS. 3A-3D in that the dilator portion 56 has been eliminated. Whereas inclusion of the dilator portion 56 may in some situations facilitate initial catheter implantation and may assist the early retention of the catheter distal end in the organ, it is recognized that the dilator portion may not be desired in other situations. Accordingly, an alternative fixation device 134 is depicted in FIGS. 9A-9F showing a base member 136 comprising a straight tube 138 dimensioned to be affixed around the catheter 132. A sleeve 140 of porous material, substantially identical to aforediscussed sleeve 66, is affixed around tube 138.

In an exemplary use of the apparatus thus far described, an operating physician will open a sterile package containing the assembly 30 of FIGS. 2A-2C including the delivery catheter 32 (or alternative delivery catheter 132 or the like) with the insertion needle 34 preinstalled. The procedure for inserting the assembly 30 should be performed under sterile conditions in a standard procedure room, ideally equipped with imaging capabilities, such as ultrasound or fluoroscope or the like. Routine equipment should be available for subject monitoring. Emergency resuscitation equipment and appropriate drugs should also be readily available. The administration of conscious sedation and local lidocaine injection is recommended, but the need will be determined by the operating physician.

In an example use where the organ is a superficial inguinal lymph node (SILN), the operating physician should utilize ultrasound or other appropriate imaging to thoroughly evaluate each subject's SILN. The SILN should be selected on the basis of size and location, and should be of sufficient size in at least one plane of imaging to accommodate the catheter during its intended use. Following insertion, the catheter distal tip should be located deep enough in the lymph node to prevent easy dislocation. The lymph node should also be located to 1) allow access using the catheter insertion procedure, 2) experience a limited range of motion during normal physical activity for the subject, and 3) provide sufficient space for adhesive anchorage of the catheter proximal end on the overlying skin.

An exemplary catheter insertion procedure includes the following steps:
1) The patient's inguinal region is depilated, draped, and prepared for sterile procedure.
2) A small incision 152 (e.g., 6-8 mm) is created in the skin, e.g., approximately 2 cm from the lymph node 154 (FIG. 10A).
3) The catheter assembly 155, including catheter 156 and insertion needle 157, is removed from its protective sleeve 158 (FIG. 10B) and the insertion needle is flushed with saline.
4) The catheter 156 with insertion needle 157 is inserted (FIG. 10C) through the incision and subcutaneous tissue and into the organ 154, e.g., longitudinal axis of the lymph node, placing the catheter distal tip inside the node and the fixation device 160 in the subcutaneous tissue surrounding the node. The catheter entry angle may be altered to accommodate differences in lymph node location and subcutaneous fat. A sufficient length of the catheter should remain outside the incision site to provide adhesive anchoring.
5) The insertion needle 157 is carefully removed (FIG. 10D) from the catheter, the catheter is aspirated, and then filled with a priming volume, e.g., of sterile heparinized saline.
6) The catheter may be closed by end cap 150 (FIG. 10E) and, at the operating physician's discretion, the incision site may be closed around the catheter.
7) The catheter is secured beneath a sterile dressing until the organ is to be next accessed, e.g., for supplying a therapeutic agent. This sterile dressing will cover and protect the incision site and catheter proximal end between visits, and will also be replaced at each follow-up visit.

An exemplary therapeutic agent delivery procedure includes the following steps:
1) The sterile dressing is removed from the insertion site and the area should be cleaned with disinfectant (chlorhexidene gluconate or povidone iodine).
2) The cap is removed from the catheter (FIG. 10F).
3) The blunt delivery needle is removed from sterile packaging, affixed to a syringe, \flushed with saline, then inserted into the catheter (FIG. 10G).
4) A syringe containing a dose of a therapeutic agent is coupled to the delivery needle (FIG. 10H).
5) The therapeutic agent is slowly injected into the lymph node over a prescribed interval.
6) The needle is removed and air is aspirated from the catheter. A priming volume of sterile heparinized saline is injected into the catheter.
7) The catheter is recapped with a new sterile cap and secured beneath an adhesive bandage (FIG. 10I) until the next treatment.

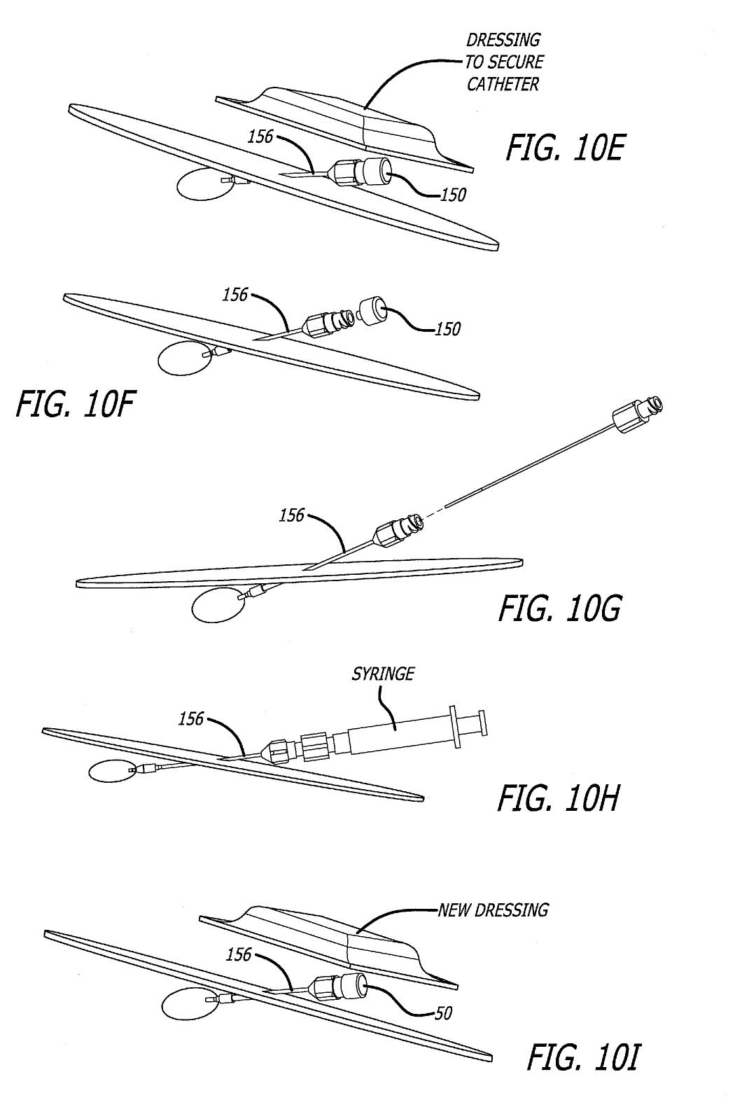

Attention is now directed to FIG. 11 which schematically illustrates another alternative embodiment of the invention. Recall in FIG. 1 that the catheter 10 is placed percutaneously, with the catheter proximal end portion 16 extending exteriorly for anchoring, e.g., by tape, to the patient's skin 18. In the embodiment of FIG. 11, the catheter 10A, which may in some embodiments carry a fixation device 22A similar to fixation devices described earlier, remains subcutaneous and has a proximal end portion 16A configured for coupling to a port 200 of a subcutaneously implanted device 202. The device 202 may include a self sealing septum 204 operably connected to an internal chamber 206 which is, in turn, in fluid communication with the lumen of catheter 10A via the port 200.

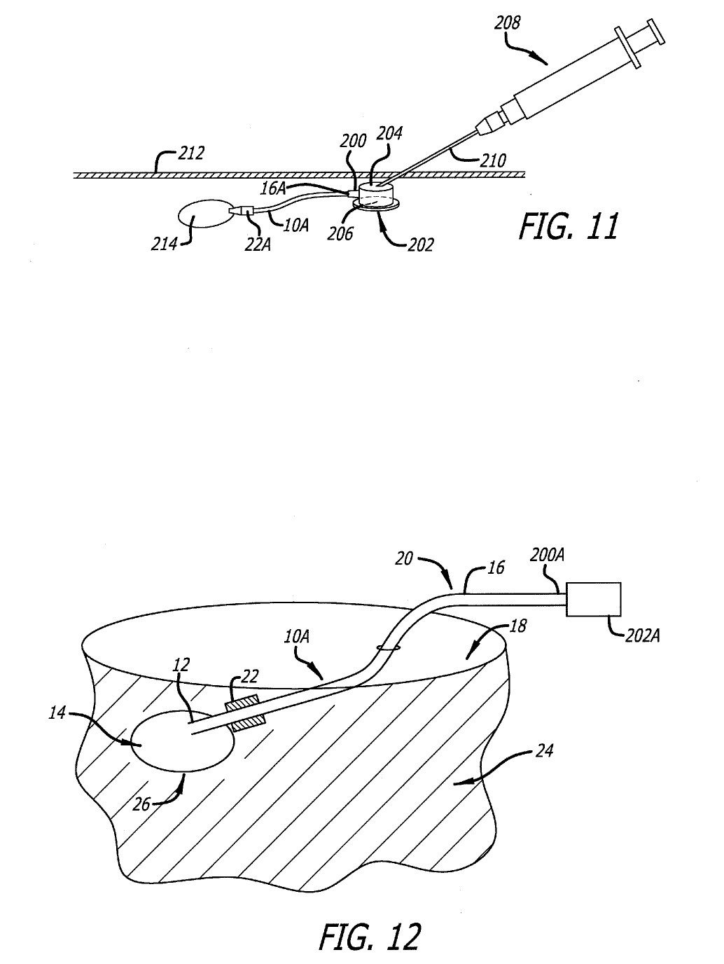

In use, a syringe 208 having a needle 210 is used to penetrate the patient's skin 212 and septum 204 to deliver material to or extract material from the organ 214, e.g., lymph node, via the catheter 10A, port 200, and the internal chamber 206 of subcutaneous device 202. In another alternative, device 202 contains long-acting or long-lasting therapeutic agent in the internal chamber 206, which is delivered in a time-released fashion through port 200 and catheter 10A. In yet another alternative (FIG. 12), device 202A is secured outside the patient's skin 18 and catheter 10A is placed percutaneously, with the proximal end attached to port 200A and distal end extending into the body organ, such as lymph node 14.

From the foregoing, it should now be clear that methods and apparatus have been disclosed for providing access such as long term repetitive or continuous access to an internal body organ. Although certain exemplary embodiments have been described in detail, it should be recognized that variations and modifications are likely to occur to those skilled in the art which fall within the intended scope of the inventions. Moreover, note that the drawings show exemplary configurations and dimensions. These configurations and dimensions are shown to fully describe exemplary embodiments of the inventions and it should be understood that they are not intended to limit the scope of the inventions as defined by the appended claims.

Although the inventions disclosed herein have been described in terms of the exemplary embodiments above, numerous modifications and/or additions to the above-described preferred embodiments would be readily apparent to one skilled in the art. It is intended that the scope of the present inventions extends to all such modifications and/or additions and that the scope of the present inventions is limited solely by the claims set forth below or later added.

Finally, with respect to terminology that may be used herein, whether in the description or the claims, the following should be noted. The terms “comprising,” “including,” “carrying,” “having,” “containing,” “involving,” and the like are open-ended and mean “including but not limited to.” Ordinal terms such as “first”, “second”, “third,” do not, in and of themselves, connote any priority, precedence, or order of one element over another or temporal order in which steps of a method are performed. Instead, such terms are merely labels to distinguish one element having a certain name from another element having a same name (but for the ordinal term) to distinguish the elements. “And/or” and “or” mean that the listed items are alternatives, but the alternatives also include any combination of the listed items. The terms “approximately,” “about,” “substantially” and “generally” allow for a certain amount of variation from any exact dimensions, measurements, and arrangements, and should be understood within the context of the description and operation of the invention as disclosed herein. Terms such as “top,” “bottom,” “above,” and “below” are terms of convenience that denote the spatial relationships of parts relative to each other rather than to any specific spatial or gravitational orientation. Thus, the terms are intended to encompass an assembly of component parts regardless of whether the assembly is oriented in the particular orientation shown in the drawings and described in the specification, upside down from that orientation, or any other rotational variation.

1. Apparatus suitable for providing access to the interior of a body organ, said apparatus including:
an elongate flexible catheter having an outer surface and an inner surface enveloping a lumen extending between a catheter proximal end and a catheter distal end; and
a sleeve of porous material mounted on at least a portion of said catheter outer surface near said catheter distal end.

2. The apparatus of claim 1 further including: a dilator fixed to said catheter outer surface having a proximal end located adjacent to said porous material sleeve distal boundary and a distal end located near said catheter distal end, said dilator having an outer peripheral surface increasing from a diameter D1 at its distal end to a diameter D2 at its proximal end where D2 is equal to or greater than a diameter D3 of said sleeve.

3. The apparatus of claim 1 further including an elongate needle configured for insertion through said catheter lumen to position the distal end of said needle beyond said catheter distal end for insertion through the peripheral wall of a body organ.

4. The apparatus of claim 3 wherein said needle distal end has a sharp tip for cutting through said organ periphery.

5. The apparatus of claim 3 wherein said needle distal end has a blunt tip for facilitating axial movement of said needle through said catheter.

6. The apparatus of claim 1 wherein said sleeve of porous material defines a distal boundary located within about 0.5 inch of said catheter distal end.

7. The apparatus of claim 1 wherein the body organ is a solid body organ.

8. The apparatus of claim 1 wherein said porous material is characterized by a pore size within a range of 50 to 200 microns and a porosity of between 60% to 95%.

9. The apparatus of claim 1 further including a device with a chamber in fluidic communication with the catheter proximal end.

10. A method for accessing the interior of a body organ, said method comprising:
providing a flexible elongate catheter having an outer surface and an inner surface enveloping a lumen extending between a catheter proximal end and a catheter distal end;
providing a layer of porous material on at least a portion of said catheter outer surface near said catheter distal end;
providing an elongate needle having a proximal end and a distal end and a sharp cutting edge located at said distal end;
implanting the distal end of said catheter in a patient's body using said needle sharp distal end, so said catheter distal end projects into the interior of a body organ with said porous material located in subcutaneous tissue outside of said body organ.

11. The method of claim 10 further comprising:
withdrawing said needle proximally from said catheter while leaving said catheter distal end projecting into said body organ; and
inserting an additional needle having a blunt distal end through said catheter lumen to extend beyond said catheter distal end into said organ.

12. The method of claim 11 wherein said additional needle includes an interior lumen extending between said additional needle proximal and distal ends, and further including:
supplying material to the proximal end of said additional needle interior lumen for delivery to the interior of said body organ.

13. The method of claim 10 wherein said step of implanting said catheter includes percutaneously extending said catheter to locate the proximal end of said catheter exteriorly of the patient's skin.

14. The method of claim 13 wherein the step of implanting the catheter includes coupling the catheter proximal end to a device with a chamber in fluidic communication with the catheter proximal end.

15. The method of claim 10 wherein said step of implanting said catheter includes coupling the proximal end of said catheter to a subcutaneous device.

16. The method of claim 15 wherein the subcutaneous device includes a self sealing septum.

17. The method of claim 10 where said porous material is characterized by a pore size within a range of 50 to 200 microns and with a porosity of between 60% to 95%

18. The method of claim 10 further comprising providing a dilator surface on said catheter outer surface distally of said porous material where said dilator surface defines a distal end located proximally of said catheter distal end;
